(19) United States
(12) Patent Application Publication
(10) Pub. No.: US 2013/0071829 A1
(43) Pub. Date: Mar. 21, 2013

(54) WAY OF OPTIMIZATION OF THE PSYCHO-PHYSIOLOGICAL CONDITION OF THE PERSON AND THE TRAINING COMPLEX

(71) Applicant: Oleg Berezhkov, Moscow (RU)

(72) Inventor: Oleg Berezhkov, Moscow (RU)

(21) Appl. No.: 13/236,540

(22) Filed: Sep. 19, 2011

(51) Int. Cl. G09B 5/04; A61M 21/02

(52) U.S. Cl. 434/319; 600/28; 600/27

(57) ABSTRACT

The invention involves training complexes and methods for optimization of the psycho-physiological condition of a person. It includes new capabilities and methods for training the skills of relaxation and activation of psycho-physiological processes. The proposed training complex consists of a training apparatus, audio-video system, training complex control system and methods for training the skills of relaxation and activation of psycho-physiological processes. Three types of training apparatus are employed: special adjustable armchair, the immersion tank and hyperbaric/hypobaric pressure chamber.

### BRIEF DESCRIPTION OF THE INVENTION

The invention improves existing training complexes intended for training skills of relaxation and activation of psycho-physiological processes. The proposed training complex consists of: a) Training device; b) Audio-video system; c) Training complex control system; d) Methods for training the skills of relaxation and activation of psycho-physiological processes.

These and other features, aspects, and advantages of the present invention will become apparent from a reading of the detailed description of the preferred embodiments of the invention (including the subjoined claims) and by reference to the attached drawings.

### BRIEF DESCRIPTION OF THE DRAWINGS

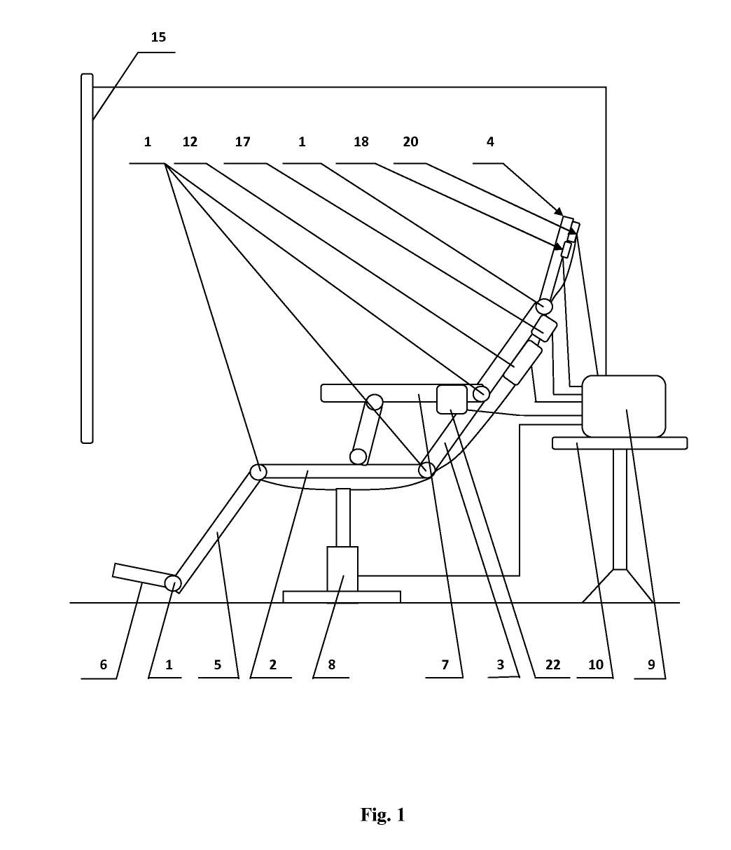

On FIG. 1—the training complex with the use of the armchair training apparatus is shown.

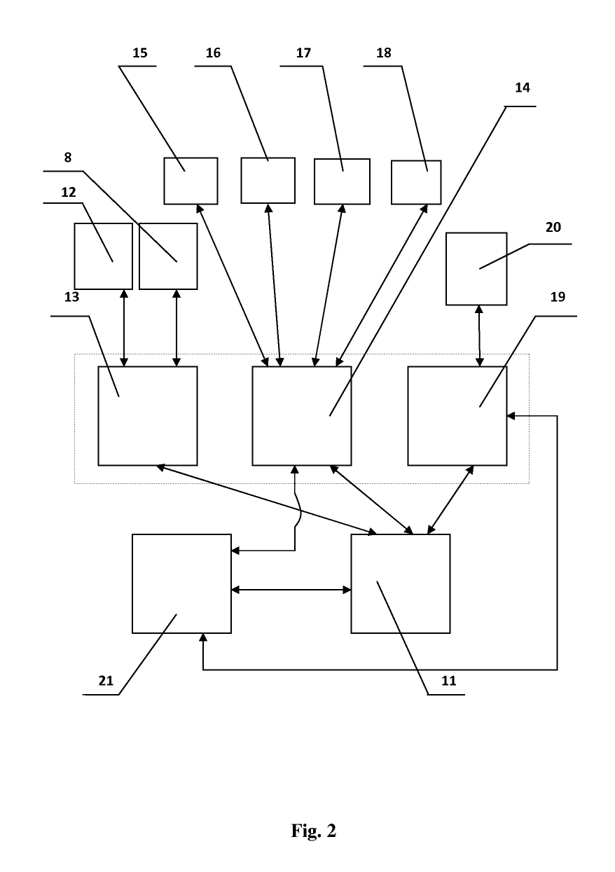

On FIG. 2—the control system of the training complex with the use of the armchair training apparatus is shown.

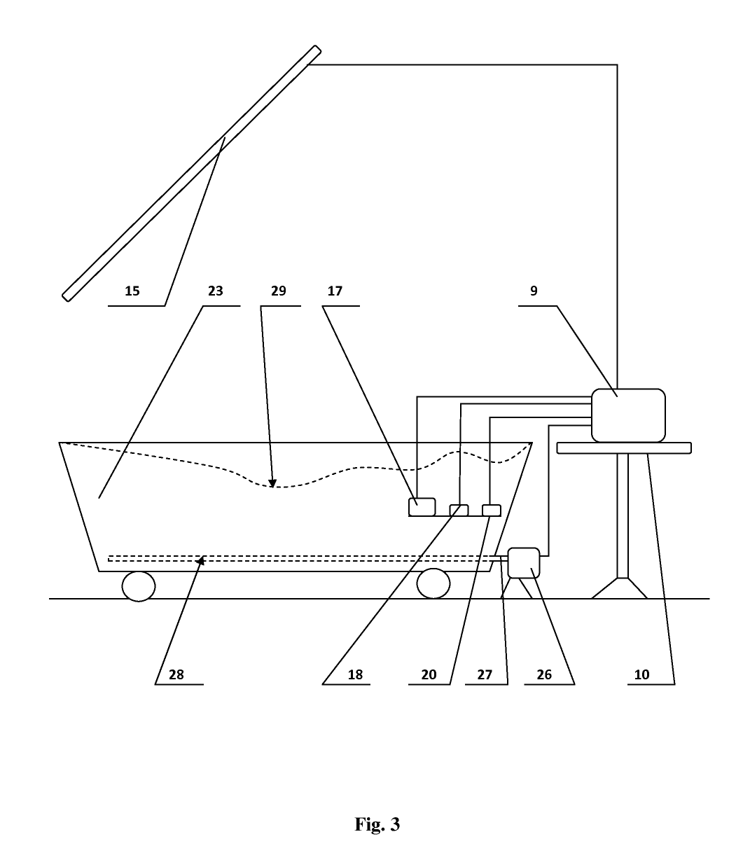

On FIG. 3—the training complex with the use of the immersion tank training apparatus is shown.

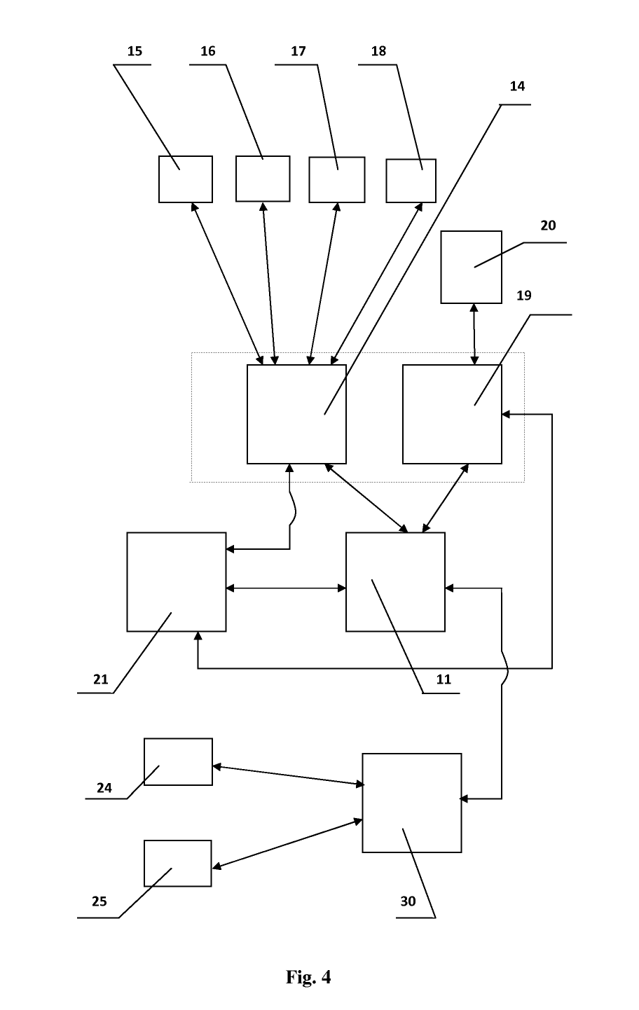

On FIG. 4—the control system of the training complex with the use of the immersion tank training apparatus is shown.

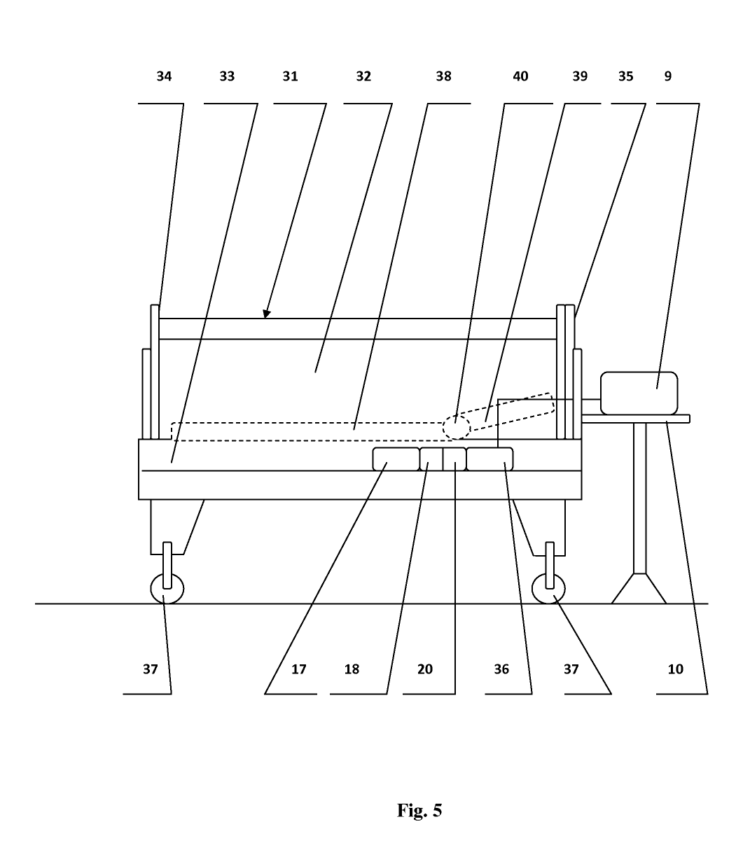

On FIG. 5—the training complex with the use of the pressure chamber training apparatus is shown.

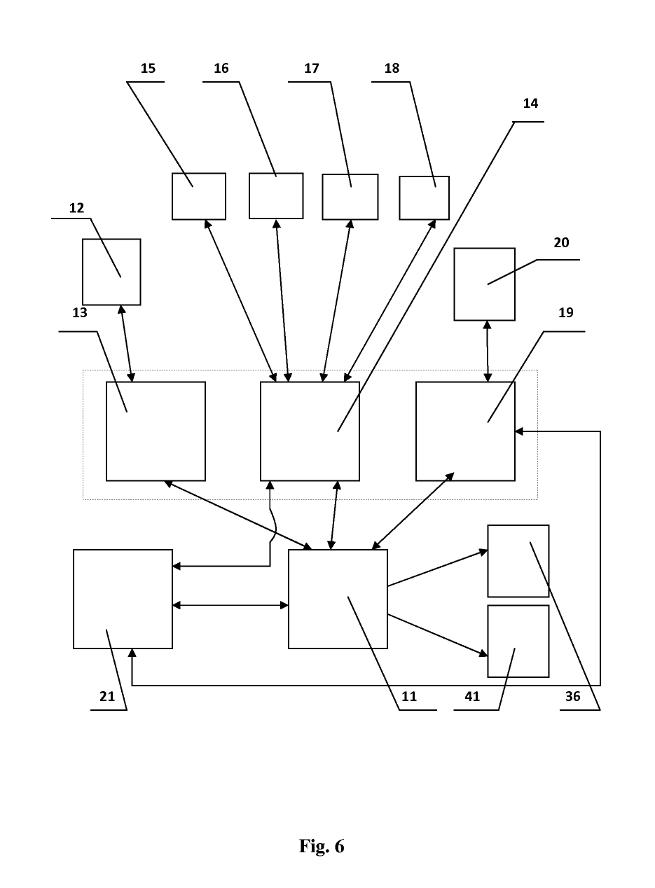

On FIG. 6—the control system of the training complex with the use of the pressure chamber training apparatus is shown.

### DETAILED DESCRIPTION OF THE INVENTION

The present invention improves existing training complexes intended for training skills of relaxation and activation of psycho-physiological processes in the following areas: a) Expansion of universality; b) Expansion of functions of the training complex; c) Increased reliability and safety; d) Increased range of capabilities. The above is achieved through the following:

(1) The method of optimization of psycho-physiological state of a person that includes the following: a) Preliminary diagnostics of the physical condition of a person; b) A person is given the individual program of training taking into account his physiological parameters; c) The person is placed on a training apparatus in the set position; d) Audio ear stimulation during a set time together with a constant control of the physical condition; e) During audio ear stimulation the additional sound stimulation on the organism is conducted with the use of the bone conduction speakers.

The ear stimulation of a person is done by the means of ear-phones or speakers. It is done in a combination with the stimulation of visual organs or olfactory organs, or simultaneously both visual and olfactory organs.

Then three types of training of relaxation and activation of psycho-physiological processes are conducted:

a) Relaxation training is given to a person with the use of partial weightlessness using the immersion tank, and then a program of training of activation of physical condition with the use of a hyperbaric pressure chamber is conducted.

b) Relaxation training is given to a person with the use of a training armchair, and then a program of training of activation of physical condition with the use of a hyperbaric pressure chamber is conducted.

c) Relaxation training is given to a person with the use of a hypobaric pressure chamber, and then a program of training of activation of physical condition with use of a training armchair is conducted.

(2) The training complex consisting of a) Training device; b) Audio-video system; c) Training complex control system. Detailed description follows.

a) The training device can be an armchair consisting of components connected with hinges with the possibility of changing its position and height adjustment with the help of a special microcontroller belonging to the overall control system.

The training device can be an immersion tank that creates the effect of partial weightlessness. It contains an elastic cover that is tightly sealing the tank along its perimeter. The tank is supplied with the system of letting water in and out as well as heating the water and is controlled by the main control system of the training complex. The immersion tank is designed to be mobile.

The training device can be a hyperbaric/hypobaric pressure chamber designed in the form of a cylinder with transparent walls placed on a massive base designed to be mobile. There is a disk-shaped door to enter. The massive base incorporates the control/display unit of the pressure chamber which is connected with the control system of the training complex. The video and the main and additional audio systems are located inside the pressure chamber. The pressure chamber is supplied with a reclining bed located in the cylinder with the possibility of a horizontal movement and the change of recline. The pressure within the chamber can be increased and decreased.

b) The training complex is supplied with the audio-video system connected with the main control system. The basic audio system consists of speakers or ear-phones. The training complex also is supplied with an additional bone conduction audio system.

c) The training complex is supplied with the control system which includes: (1) A control panel, connected through the microcontroller with the audio-video system; and (2) The monitoring system that monitors the physical condition of a subject.

The training complex also is supplied with additional bone conduction audio system that is managed by the additional microcontroller.

The control system includes a microcontroller that regulates the height and position of the armchair.

The monitoring system of the physical condition of a subject provides a feedback function to microcontrollers of the audio-video system, the main audio system, the additional audio system, as well as to the control panel.

The invention is illustrated by the attached drawings. On FIG. 1—the training complex with the use of the armchair training apparatus is shown. On FIG. 2—the control system of the training complex with the use of the armchair training apparatus is shown. On FIG. 3—the training complex with the use of the immersion tank training apparatus is shown. On FIG. 4—the control system of the training complex with the use of the immersion tank training apparatus is shown. On FIG. 5—the training complex with the use of the pressure chamber training apparatus is shown. On FIG. 6—the control system of the training complex with the use of the pressure chamber training apparatus is shown.

The optimization of a psycho-physiological condition of a person can be accomplished by using different devices. The training complexes represented in FIGS. 1-6, have been designed to provide a specific method to accomplish this and are described in the patent application.

The training complex represented on FIGS. 1 and 2 includes the training apparatus executed in the form of an armchair. It consists of components connected with hinges 1 with the possibility of adjusting the position of individual components: seat 2, the backrest 3, the headrest 4, the calf support 5, feet support 6 and armrests 7. The armchair is supplied with height adjustment mechanism 8. All components have the possibility of changing the angle of a slope using hinges 1 as well as distances between them using guiding rails and fixing pins to select a position (not illustrated). Controlling system 9 of the training complex is located on the stand 10 and includes the following blocks: operating panel 11 connected with (a) microcontroller 13, controlling height adjustment mechanism 8 and position adjustment mechanism 12 of the armchair; (b) microcontroller 14 controlling audio-video system 15, video system 16, audio-system 17 and ear-phones 18; (c) microcontroller 19 controlling additional audio system 20 with bone conduction speakers; and (d) control system 21 of physical condition of the visitor. Controlling system 21 of physical condition of the subject is connected for the feedback function with microcontrollers 14 and 19 and with operating panel 11.

In addition, the training complex can be equipped with massage elements built in certain zones of the armchair as well as with the system providing aromatic air supply to the client that can be either independent or connected with the training complex. Armrests 7 can be supplied with position adjustment mechanism 22.

The training complex represented on FIGS. 3 and 4 includes mobile immersion tank 23 connected with water heating and supply systems 24 and 25 located in portable block 26 connected with punched pipe 28 by means of a hose 27. Immersion tank 23 is supplied with drainage system (not shown). Punched pipe 28 located at the bottom of the tank 23 is intended for water delivery as well as for massage procedures. Elastic cover 29 is tightly fixed along the perimeter of the tank 23 at its top surface and is intended to be used for a “dry immersion” method. Controlling system 9 of the training complex can be put on the stand 10 or mounted on the tank 23. It includes the following blocks: operating panel 11 connected with (a) microcontroller 14 controlling audio-video-system 15, video-system 16, audio-system 17 and ear-phones 18, (b) microcontroller 19 controlling additional audio system 20 with bone conduction speakers (c) microcontroller 30 controlling water heating and supply systems 24 and 25 and (d) control system 21 of physical condition of the visitor. Controlling system 21 of physical condition of the subject is connected with microcontrollers 14 and 19 and with operating panel 11 to provide the feedback function.

The training complex represented on FIGS. 5 and 6, includes a pressure chamber consisting of cylinder 31 with transparent walls 32, located on massive base 33 with metal ring flanges 34. One of the flanges 34 is connected with the disk-shaped door 35. On the external panel of the massive base 33 control/display unit of the pressure chamber 36 is mounted. It is connected with controlling system 9 of a training complex. Massive base 33 is executed in the form of a cart with wheels 37 for moving indoors. The pressure chamber is supplied with the reclining bed located in the cylinder 31 with the possibility of horizontal movement. Its parts are connected with hinge 40 to allow the possibility of changing the angle of the slope of the headrest 39. The pressure chamber is executed with the possibility of increasing and decreasing pressure in cylinder 31 by means of the control/display unit 36. Controlling system 9 of a training complex can be located on separate support 10 or installed on the massive base 33. Controlling system 9 includes the following blocks: operating panel 11 connected with (a) microcontroller 13, intended for automatic resetting the angle of slope of headrest 39 by means of its adjusting mechanism 12, (b) microcontroller 14 controlling audio-video-system 15, video-system 16, audio-system 17 and ear-phones 18, (c) microcontroller 19 controlling additional audio system 20 with bone conduction speakers. Operating panel 11 is connected with the two-way system 41 and with controlling system 21 of physical condition of the subject. Controlling system 21 of the physical condition of the subject is connected with microcontrollers 14 and 19 and with operating panel 11 to provide the feedback function.

The training complex is used in training salons, fitness clubs, and in relaxation centers. Training apparatus can be installed in rooms with other types of training machines, or if necessary, in separate rooms. Control system 9 is capable working in different regimens: physical relaxation, psychological relaxation, activation of physical activity, activation of cerebral activity and others.

To each of the mentioned regimens corresponds a certain frequency of the audio-system, a certain rhythm of sounding, video program, air composition, water temperature in the immersion tank 23 and elevated or reduced pressure in the pressure chamber.

Utilizing the described above training complex it is possible to provide the following method of optimization of a psycho-physiological condition of a person. The initial physical condition of a client is diagnosed before starting the training by measuring the basic physiological parameters, such as arterial pressure, a pulse rate and others. The measured parameters are entered into the memory of controlling system 21 of the physical condition of the subject. After interview with the trainer the client is given an individual program of training taking into account his physiological parameters. Depending on the chosen program of training the client is sent to a particular training apparatus.

Individual training includes relaxation training consisting of audio program of guided meditation in combination with special effects that enhance relaxation. Audio programs can be carried through audio system 20, or through ear phones, or through audio-video system 15. Audio program includes the segment directed at breath and heart pulse optimization. The client is instructed to tune the breath to a particular audio rhythm. This could be, for example, the rhythm of ocean waves, of metronome, rhythmic music etc. Supplied cooled oxygenated air imitates fresh ocean breeze. Aromatherapy and certain sounds of nature are synchronized with guided information about forest, ocean, rain etc. Similar special visual effects like for example imitation of dawn and sunset are used to enhance the relaxation effect of a guided meditation.

When using the training complex represented on FIGS. 1 and 2, armchair control takes place, by changing the angles of slope of seat 2, backrests 3, headrest 4, calf support 5 and feet support 6. The height of seat 2 is adjusted. Microcontroller 13 in the controlling system 9 of the training complex allows to conduct these adjustments in the automatic regimen according to the chosen program of training. The most optimum program of training includes all available ways to stimulate subject, for example, visual and sound through audio-video-system 15 in combination with additional audio-system 20 equipped with the bone conduction speakers. Also it is possible the utilization of the air aroma system (not shown), in combination with the increase or decrease of oxygen in the air mixture provided to the subject. Audio stimulation can be carried out through individual ear-phones 18 or through general audio-system 17.

A bone conduction speaker of audio-system 20 is fixed to the helmet or to the special rim covering the head of the subject designed to have close contact with the bones of the skull.

In a case when the subject prefers to sit or lie down with eyes closed audio-video-system 15 and video-system 16 are not used and stimulation is conducted using all other available subsystems.

Training is conducted under the constant control of physical and physiological condition of the subject.

When immersion tank 23 (see FIG. 3) is used for conducting the training a certain water temperature is selected and the subject is put on elastic cover 29 that separates the person from water. The person appears to be freely “suspended” in water and the pressure rendered on various parts of a body is counterbalanced. The effects of gravity are reduced, the tone and power qualities of extensor muscles go down, the activity of motor control mechanisms and the activity of the basic muscular proprioceptive system goes down. The volume of liquid released from the organism increases, the muscle tone decreases and heart works half as hard. These almost perfect conditions for relaxation trainings allow the heightened effect of the basic audio-systems 17, 18 and 20 on the human body. Conducting a massage of the body of the subject during training which is done by means of water sprays coming upwards toward elastic coverlet 29 from the punched pipe 28 allows even more effective relaxation training.

The training complex with the use of the pressure chamber which is designed in the form of cylinder 31 with transparent walls 32 allows conducting different types of trainings.

The program of training of activation of physical condition is conducted by raising the pressure inside the hypobaric chamber to a certain set of parameters. The relaxation training is conducted by reducing the pressure inside the chamber.

All trainings include sound stimulation utilizing the basic audio-system 17 and additional audio-system 20 with the bone conduction speaker. By positioning video-system 16 in the subject's zone of vision outside transparent cylinder walls 32 of hypobaric chamber 31, the effect of training is improved. Using control/display unit 36 of the pressure chamber, different air mixtures with increased or decreased concentration of oxygen as well as natural aromatics are supplied to the cylinder 31.

Pressure chamber is used in the following way. (1) Door 35 of cylinder 31 gets opened, (2) The reclining bed is rolled out. The reclining bed consists of mattress 38 and headrest 39 connected with hinges, so it is possible to change the angle of slope of headrest 39 around hinge 40. (3) The subject is placed on the reclining bed and is rolled into the pressure chamber. (4) The training program is started with the help of operating panel 11. There is a continuous two-way communication with the subject through 41 and control of his condition through 21. A special complex of physical exercises has been developed to be performed in the hypobaric chamber to strengthen efficiency of the training directed at activation of physical activity.

The efficiency of the described above ways of optimization of a psycho-physiological condition of a person can be strengthened by using special methods of breath, for example holotropic, static and dynamic meditations as well as special complexes of exercises, for example yoga asanas and pranayamas. A client is offered the possibility of including into the training program these additional exercises since they increase the production of such important hormones as endorphins as well as corticosteroids that provide anti-inflammatory action in the body.

Combined use of different training apparatus, for example, relaxation training in the immersion tank with the subsequent activation of physical condition in the pressure chamber, provides synergistic effect and allows to reach great and stable positive results in the psycho-physiological condition of the person.

The training complex is designed to provide a high level of safety to a client. Control system 21 monitors condition of the client and can safely shut down the training complex any time the undesirable physiological parameters of the client get into a dangerous zone.

The technical results that have been reached by this invention are the following: (1) The expansion of universality of the training complex designed to train the skills of relaxation and/or activation of psycho-physiological processes; (2) Increased efficiency of optimization of psycho-physiological condition of the person; (3) The expansion of functions of a training complex; (4) Increased reliability and safety.

1. A method of optimization of the psycho-physiological condition of the person, that includes the following: a) preliminary diagnostics of the physical condition of a person; b) a person is given the individual program of training taking into account his physiological parameters c) he is placed on a training apparatus in the set position; d) audio ear stimulation during a set time together with a constant control of the physical condition; e) during audio ear stimulation the additional sound stimulation on the organism is conducted with the use of the bone conduction speakers.

2. The method of optimization of the psycho-physiological condition of the person as described in item 1 and is different in that it is conducted in combination with the stimulation of visual organs or olfactory organs, or simultaneously of both visual and olfactory organs.

3. The method of optimization of the psycho-physiological condition of the person as described in items 1 and 2 and is different in that it includes relaxation training consisting of audio program in combination with special effects (like turning on and off color lights, supply of cold and then warm air) that enhance a guided meditation designed for relaxation.

4. The method of optimization of the psycho-physiological condition of the person as described in item 1 and is different in that it is conducted in combination with the effect of partial weightlessness using the immersion tank as part of the training apparatus.

5. The method of optimization of the psycho-physiological condition of the person as described in item 1 and is different in that it is conducted in the isolated environment within the pressure chamber with the increased or decreased air pressure.

6. The method of optimization of the psycho-physiological condition of the person as described in item 1 and is different in that first the person is given a relaxation training using the state of weightlessness in the immersion tank and then the training program of activation of the physical condition using the hyperbaric pressure chamber.

7. The method of optimization of the psycho-physiological condition of the person described in item 1 and is different in that first the person is given a relaxation training using special training armchair and then the training program of activation of physical condition using the hyperbaric pressure chamber.

8. The method of optimization of the psycho-physiological condition of the person described in item 1 and is different in that first the person is given a relaxation training using the hypobaric pressure chamber and then the program of training of activation of physical condition using special training armchair.

9. The training complex consisting of the training device, the audio system and the control system of the training complex. The control system consists of control panel connected with an audio system through microcontroller and control system that monitors the physical condition of the visitor. The basic audio system consists of speakers or ear-phones. The training complex also is supplied with the additional audio system utilizing the bone conduction speaker and additional microcontroller that controls additional audio system.

10. The training complex as described in 9 but more specifically the training apparatus is an armchair consisting of components connected with hinges with the possibility of adjusting the position and height of individual components. The control system includes a microcontroller that regulates the height and position of the armchair

11. The training complex as described in 9 in addition is supplied with the audio-video-system which is connected to the control system.

12. The training complex as described in 9 but more specifically the monitoring system of the physical condition of a subject is linked through the feedback connection with microcontrollers of audio-video-system, the main audio-system, additional audio-system, as well as the control panel.

13. The training complex as described in 9 but more specifically the training apparatus is a stationary immersion tank containing the water flow supply and water heating systems. The tank includes the elastic cover that is tightly fixed along the perimeter of the tank and its top surface is used for “dry immersion” procedures. There is a punched pipe at the bottom of the tank intended for water delivery as well as for massage procedures. The water supply and heating systems are managed by a special microcontroller belonging to the overall control system of the training complex.

14. The training complex as described in 13 and is different in that the immersion tank is mobile.

15. The training complex as described in 9 but more specifically the training apparatus is a pressure chamber. The pressure chamber is executed in the form of the cylinder with transparent walls, located on the massive basis and is supplied with the face metal ring flanges, thus at least one of the flanges is connected with adjacent to it disk-shaped door, and the massive basis is supplied with the control/display unit of the pressure chamber, which is connected with the control system of the training complex. The main and additional audio systems are located inside the pressure chamber.

18. The pressure chamber as described in 15 and is different in that the massive basis of the cylinder is mobile.

19. The pressure chamber as described in 15 and is different in that it is supplied with the reclining bed located in the cylinder with the possibility of a horizontal movement and the change of recline.

20. The pressure chamber as described in 15 and is different in that it is executed with the possibility of increasing and decreasing the pressure in the cylinder.
